(19) United States
(12) Patent Application Publication
(10) Pub. No.: US 2016/0206780 A1
(43) Pub. Date: Jul. 21, 2016

(54) Matrix Scaffold for Three-Dimensional Cell Cultivation, Methods of Construction Thereof and Uses Thereof

(71) Applicant: SUZHOU CANCERCELL BIOTECHNOLOGY CO. LTD, Jiangsu (CN)

(72) Inventor: Shouli WANG, Suzhou City, Jiangsu (CN)

(21) Appl. No.: 14/774,086

(22) PCT Filed: Aug. 15, 2014
(86) PCT No.: PCT/CN2014/000771

§ 371 Date: Sep. 9, 2015

(51) Int. Cl. A61L 27/26; A61L 27/20; C12N 5/077; A61L 27/56; C12N 5/00; C12N 5/09; A61L 27/22; A61L 27/58

(52) U.S. Cl. CPC A61L 27/26; A61L 27/227; A61L 27/20; A61L 27/58; A61L 27/56; C12N 5/0062; C12N 5/0693; C12N 5/0658; A61L 2430/34; A61L 2430/40; C12N 2513/00; C12N 2533/50

(57) ABSTRACT

This invention provides a matrix scaffold for three-dimensional (3D) cell cultivation, a method of its construction, and its uses. The matrix scaffold results from a crosslinking reaction of a fibroin-like substance prepared by a specific method from silk cocoons or filaments, chitosan and crosslinking agents. This matrix scaffold is more advantageous in terms of degradation stability and benefits to 3D cell cultivation over existing matrix scaffolds. In addition, it can replace extracellular matrices (ECMs) or tissue and organ matrices and is further for use for the in-vitro differentiation and proliferation of cells, tissue and organ reconstruction and antitumor drug screening.

### TECHNICAL FIELD

The present invention relates to a matrix scaffold for three-dimensional (3D) cell cultivation, a method of its construction and its uses. The scaffold can replace extracellular matrices (ECMs) or tissue and organ matrices and is further for use for the in-vitro differentiation and proliferation of cells, tissue and organ reconstruction and antitumor drug screening.

### BACKGROUND

Frequent reports of side effects arising recently from the clinical application of a great number of biological targeting drugs have attracted more and more attention. A major cause of why preclinical studies have failed in finding these side effects is considered to be the distinctions between the real in-vivo environment and those provided by the models used in the studies. Creating a research model that can provide an environmental closely approximating the real in-vivo environment of the human body is perhaps a key solution for this problem.

Cells are common research tools, and the current cell-level studies are basically carried out with cells two-dimensionally (2D) cultured in in-vitro environments, which, however, hardly well model the in-vitro cells whose growth is all supported by a certain kind of matrix (irrespective of adherently or non-adherently growing cells).

In recent years, 3D cell cultures have gradually become the focus of research efforts in this field. These methods utilize a variety of materials and approaches to allow cells to grow in 3D space in a manner that is closer to cell growth in the in-vivo environment into a structure resembling an in-vivo tissue in terms of physiological functions. Depending on how cells are cultured, the commonly used existing 3D cell cultures are mainly classified into dynamic and static ones. Dynamic cultures mainly include rotary bioreactor culture systems and rotary cell culture systems. However, while these technologies allow better cellular performance by means of mechanical stimulation, the higher requirements make them less possible for wide application. Static cell cultures involve seeding cells directly onto a 3D carrier and allowing them to grow without being affected by any physical disturbance. The common categories of such technologies include spontaneous cell aggregation, covered matrices, preset matrices and microcarriers, in which the preset matrices can provide a scaffold environment closely resembling the in-vivo environment for cell growth and are associated with simple operations. The performance of such matrices depends on their materials.

Sources of matrices commonly used at present are animal gels such as, for example, collagen I, gelatin and extracellular matrix extracts, which are expensive and contain many unknown components.

CN101445971A and CN103418029A disclose fibroin-chitosan composite nanofibers for artificial ECMs, which are made from fibroin and chitosan and can provide an optimum artificial physiological environment for cell growth and tissue reconstruction. However, the nanofibers all have the deficiency that their manufacture involves the preparation of a fibroin solution which is labile and hence makes the nanofibers unsuitable for industrial applications.

CN102010601A, CN101624472A and CN101624473A disclose materials for hepatocyte-specific large-porous microcarrier scaffolds, which are made from fibroin and galactosylated chitosan under the action of a cross-linking agent and are suitable for large-scale cultivation of hepatocytes. However, these materials all have a number of deficiencies, such as complex steps in their preparation (requiring chitosan to successively undergo glycosylation, cross-linking and mesh screen filtering) and limited use due to their large-pore nature.

CN102942660A discloses a bio-crosslinked natural 3D nanocomposite gel scaffold which results from in-situ free-radical polymerization of an acrylamide monomer, inorganic nano-clay, a biopolymer and Genipi as a bio-crosslinking agent in water and can be used in medical transplants, drug delivery and cell cultivation. However, the scaffold also has a number of deficiencies. One deficiency is that the acrylamide monomer, the dominant material, tends to decompose at high temperatures and release irritating gases. Another deficiency is that the acrylamide monomer will produce acrylic acid content that is highly toxic to cells in acid or alkaline environments.

CN103418029A discloses a porous fibroin-chitosan composite scaffold made from fibroin and chitosan. This scaffold also has the deficiency that the composite is labile, making the scaffold unsuitable for industrial production and limited in application.

Chinese patent document CN102952279A discloses a method of preparing a hydrogel by reacting a methyl vinyl ether/maleic acid copolymer with a crosslinking agent and its use in tumor tissue cultures. However, the effect of the great content of cell growth factors (e.g., epidermal growth factors, fibroblast growth factors and tissue plasminogen activators) in the hydrogel matrix on cell cultivation remains uncertain.

### SUMMARY OF THE INVENTION

In order to overcome the above described deficiencies of the conventional technologies, the present invention provides a matrix scaffold for 3D cell cultivation. These matrix scaffolds resemble natural endogenous scaffolds and can be used for the in-vitro proliferation of cells that are difficult to be cultured, for tissue and organ reconstruction and antitumor drug screening.

In one aspect, the present invention relates to a matrix scaffold for 3D cell cultivation, resulting from a crosslinking reaction of a fibroin-like substance, chitosan and crosslinking agents, wherein the fibroin-like substance is prepared by obtaining a fibroin powder through subjecting silk cocoons or silk filaments to degumming, dissolution, dialysis and drying processes and performing the following steps on the fibroin powder:

1) dissolution of the fibroin powder in a lithium bromide (LiBr) solution;

2) dialysis of the fibroin solution resulting from step 1) using a dialysis bag with a cutoff molecular weight of 3,500 Daltons; and

3) concentration of the fibroin solution that has been dialyzed in step 2) by positioning the dialysis bag containing the solution in a polyethylene glycol 6000 powder, centrifugation of the concentrated solution, and obtainment of the supernatant as the fibroin-like substance.

Preferably, the crosslinking agents are 1-ethyl-3-(3-dimethylaminopropyl)carbodiimide (EDC) and N-hydroxysuccinimide (NHS).

The matrix scaffold can be obtained through gradient freezing of a product of the reaction of the fibroin-like substance, chitosan and crosslinking agents, wherein the gradient freezing includes:

1) obtaining a precursor 3D scaffold by subjecting the product to pre-freezing in a −20° C. refrigerator for 12-48 h, then freezing in a −80° C. refrigerator for 12-48 h, and then freezing and drying in a freezing dryer for 24-72 h; and

2) immersing the precursor 3D scaffold obtained in step (1) in anhydrous methanol mixed with a 10% sodium hydroxide solution (in a ratio by volume of 1:1) for 12-48 h, followed by rinsing with deionized water and drying for 24-72 h in the freezing dryer.

Various conformations and pore sizes of the matrix scaffold are possible by adjusting amount(s) of the fibroin-like substance, chitosan and/or crosslinking agents.

Preferably, the matrix scaffold is prepared by a method comprising the steps of:
A. preparation of a solution of a fibroin-like substance, including:
   1) cutting silk cocoons into small pieces and boiling the pieces in a 0.5% sodium carbonate (Na₂CO₃) solution 2-3 times, followed by rinsing with deionized water and drying;
   2) dissolving, with stirring, the dried fibroin resulting from step 1) in a boiling 50% calcium chloride (CaCl₂) solution and filtering the whole after it is cooled;
   3) obtaining a fibroin solution by dialyzing the filtrate contained in a dialysis bag against deionized water and subsequently obtaining a fibroin powder by subjecting the fibroin solution packaged in a freezer bag successively to freezing in a −20° C. refrigerator and then in a −80° C. refrigerator and drying in a freezing dryer;
   4) weighing 10 g of the fibroin powder obtained in step 3) and dissolving it in a 9 M LiBr solution, wherein the dissolution is facilitated by a stirring action at the room temperature;
   5) cooling the fibroin solution resulting from step 4) to the room temperature and then dialyzing it in a dialysis bag with a cutoff molecular weight of 3,500 Daltons for 2-4 days in order to remove low-molecular substances contained in the fibroin solution; and
   6) concentrating the fibroin solution resulting from step 5) by positioning the dialysis bag containing the solution in a polyethylene glycol 6000 powder, centrifuging the concentrated solution and collecting the supernatant as the solution of the fibroin-like substance,
B. preparation of a chitosan solution, including:
   1) preparing a 1% glacial acetic acid solution by diluting 1 mL of glacial acetic acid to 100 ml and adjusting a pH of the solution to 4.6; and
   2) dissolving an amount of chitosan (with a deacetylation of >90%) in the glacial acetic acid solution, thereby forming the chitosan solution, and
C. formation of a crosslinked scaffold, including
   1) mixing the solution of the fibroin-like substance prepared in step (1) with the chitosan solution prepared in step (2);
   2) immersing the mixture in a 95% ethanol aqueous solution containing 50 mmol/l of EDC and 18 mmol/l of NHS and maintaining the crosslinking reaction at 4° C. for 24 h;
   3) obtaining a precursor 3D scaffold by subjecting a product of the reaction in step 2) to pre-freezing in a −20° C. refrigerator for 24 h, then freezing in a −80° C. refrigerator for 24 h, and then freezing and drying in a freezing dryer for 48 h; and
   4) obtaining the matrix scaffold for 3D cell cultivation by immersing the scaffold obtained in step 3) in anhydrous methanol mixed with a 10% sodium hydroxide (NaOH) solution (in a ratio by volume of 1:1) for 24 h, followed by rinsing thrice with deionized water and drying for 48 h in the freezing dryer.

Preferably, the solution of the fibroin-like substance has a concentration of 1-5%, and the chitosan solution has a concentration of 1-5%.

In another aspect, the present invention relates to use of the matrix scaffold for 3D cell cultivation as defined above for the in-vitro differentiation and proliferation of cells, reconstruction of tissues and organs, or screening of anticancer drugs.

Preferably, the use is for the cultivation of stem cells, engineering of tumor microenvironments, screening of antitumor drugs, or engineering of tissues and organs.

Preferably, the use is for the in-vitro differentiation of myoblasts isolated from embryonic tissues or tumor associated macrophages (TAMs) or tumor-associated fibroblasts (TAFs) isolated from tumor tissues.

In a further aspect, the present invention relates to a method of in-vitro cell proliferation, which includes using the matrix scaffold as defined above as a scaffold for three-dimensional cell cultivation.

As described above, the matrix scaffold according to the present invention results from a crosslinking reaction of the fibroin-like substance produced in the manner as described above, chitosan and the crosslinking agents and is possible to be made with different pore sizes and/or in different conformations by means of adjusting the concentrations and ratios of the fibroin-like substance/chitosan solution and the crosslinking agents.

In a further aspect, the present invention also relates to a method of preparing the matrix scaffold as defined above. One specific embodiment of this method includes the steps of:
A. preparation of a 1-5% fibroin solution, including:
   (1) cutting silk cocoons (or silk filaments) into small pieces and boiling the pieces in a 0.5% Na₂CO₃ solution 2-3 times, following by rinsing twice with deionized water and drying;
   (2) adding the dried fibroin to a boiling 50% CaCl₂ solution, stirring the solution, and filtering the whole after it is cooled;
   (3) obtaining a fibroin solution by dialyzing the filtrate contained in a dialysis bag against deionized water and subjecting the fibroin solution packaged in a freezer bag successively to freezing in a −20° C. refrigerator and then in a −80° C. refrigerator and drying in a freezing dryer;
   (4) weighing 10 g of the fibroin powder and dissolving it in a 9 M LiBr solution, wherein the dissolution is facilitated by a stirring action at the room temperature;
   (5) cooling the fibroin solution to the room temperature and then dialyzing it in a dialysis bag with a cutoff molecular weight of 3,500 Daltons for 3 days, in order to remove low-molecular substances in the fibroin solution;
   (6) concentrating the fibroin solution by positioning the dialysis bag containing the solution in a polyethylene glycol 6000 powder, centrifuging the concentrated solution and collecting the supernatant; and
   (7) calculating a concentration of the fibroin solution according to: fibroin solution concentration (%)=(M3−M1)/(M2−M1)×100%, where M1 denotes the weights of three labeled weighing bottles measured after they are cleaned, dried in a 60° C. constant temperature oven and sufficiently cooled, M2 denotes the weights of the weighing bottles after they are each filled with 10 ml of the fibroin solution, and M3 denotes the weights of the weighing bottles filled with the fibroin solution after they are heated in the 60° C. constant temperature oven for 12 h and then cooled,
B. preparation of a 1-5% chitosan solution, including:
   (1) preparing a 1% glacial acetic acid solution by diluting 1 mL of glacial acetic acid to 100 ml and adjusting a pH of the solution to 4.6; and
   (2) dissolving 3-5 g of chitosan (with a deacetylation of >90%) in the glacial acetic acid solution, and
C. formation of a crosslinked scaffold, including:
   (1) mixing the fibroin solution prepared in step 1 with the chitosan solution prepared in step 2;
   (2) immersing the mixture in a 95% ethanol aqueous solution containing 50 mmol/l of EDC and 18 mmol/l of NHS and maintaining the crosslinking reaction at 4° C. for 24 h;
   (3) obtaining a precursor fibroin/chitosan scaffold by subjecting a product of the reaction to pre-freezing in a −20° C. refrigerator for 24 h, then freezing in a −80° C. refrigerator for 24 h, and then freezing and drying in a freezing dryer for 48 h; and
   (4) immersing the precursor scaffold in anhydrous methanol mixed with a 10% NaOH solution (in a ratio by volume of 1:1) for 24 h, followed by rinsing thrice with deionized water, drying for 48 h in the freezing dryer and microscopic examination.

Preferably, the use of the matrix scaffold according to the present invention is for the in-vitro differentiation of myoblasts isolated from embryonic tissues or TAMs or TAFs isolated from tumor tissues.

### BENEFITS OF THE INVENTION

According to the present invention, a natural fibroin-like substance can be obtained from silk cocoons or filaments by means of a specific extraction process. In addition, this fibroin-like substance can be crosslinked with natural chitosan in the presence of the non-toxic crosslinking agents EDC and NHS to produce a 3D scaffold of which the conformation closely resembles that of natural endogenous scaffolds. Therefore, once cells are seeded on the 3D scaffold, a microenvironment for cell growth can be rapidly created to which the cells can be quickly adapted and exert their normal physiological functions. Major benefits of the present invention are that:

1. the fibroin-like substance obtained from silk cocoons or filaments and chitosan have both been proven to be highly biocompatible and non-toxic and can provide a number of advantages over animal gels, such as, for example, easy obtainability and low cost;

2. in accordance with the fact that the growth of most mammalian cells, both in vivo and in vitro, must be based on their adherence to certain kinds of scaffolds, the 3D scaffold according to the present invention is suitable for use in the cultivation of both adherent and non-adherent cells; and, moreover, the 3D scaffold can avoid the contact inhibition effect associated with the use of a 2D scaffold and thus enables improved reliability of findings in in-vitro cellular research carried out in the scaffold, which can more objectively reflect physiological states of the cells in their life activities;

3. adjustments in concentrations and ratios of the fibroin-like substance/chitosan solution and crosslinking agents can result in various conformations and pore sizes of the 3D matrix scaffold, which can be selected to be compatible with structural characteristics of in-vivo tissues where the cells to be cultured should grow, as well as with the requirements for in-vitro cultivation of primary cells from different tissue sources, thus making the matrix scaffold possible to find extremely wide application, for example, in the in-vitro proliferation of cells that are difficult to be cultured, tissue and organ reconstruction and antitumor drug screening;

4. the 3D cell culture matrix scaffold according to the present invention can assume conformations that are closely similar to those of fibrous tissues in the in-vivo microenvironment for cell growth, so that primary isolated cells cultured in this scaffold will have rapid adaptation to this in-vitro environment and similar biological behaviors as in the in-vivo environment, thereby reducing deviations between in-vitro and in-vivo test results and creating significant social benefits; and

5. it provides a novel approach for research in 3D cell cultivation and will act as a key facilitator for the cultivation of stem cells, engineering of tumor microenvironments, screening of antitumor drugs, engineering of tissues and organs, etc.

### BRIEF DESCRIPTION OF THE DRAWINGS

FIG. 1 shows optical microscope images and scanning electron microscope (SEM) images of matrix scaffolds prepared in Example 1 of the present invention.

FIG. 2 shows the differentiation (myotube formation rate (MFR)) of primary myoblasts in a matrix scaffold prepared in Example 1 of the present invention.

FIG. 3 shows the proliferation (cell cycle) of primary myoblasts in a matrix scaffold prepared in Example 1 of the present invention.

FIG. 4 shows the growth (MTT) of TAMs in a matrix scaffold prepared in Example 1 of the present invention.

FIG. 5 shows the growth (MTT) of TAFs in a matrix scaffold prepared in Example 1 of the present invention.

FIG. 6 shows a degradability comparison of a matrix scaffold constructed in accordance with the present invention and a matrix scaffold made with conventional fibroin.

FIG. 7 shows a cell proliferation ability comparison of a matrix scaffold constructed in accordance with the present invention and a matrix scaffold made with conventional fibroin.

Findings from these figures are:

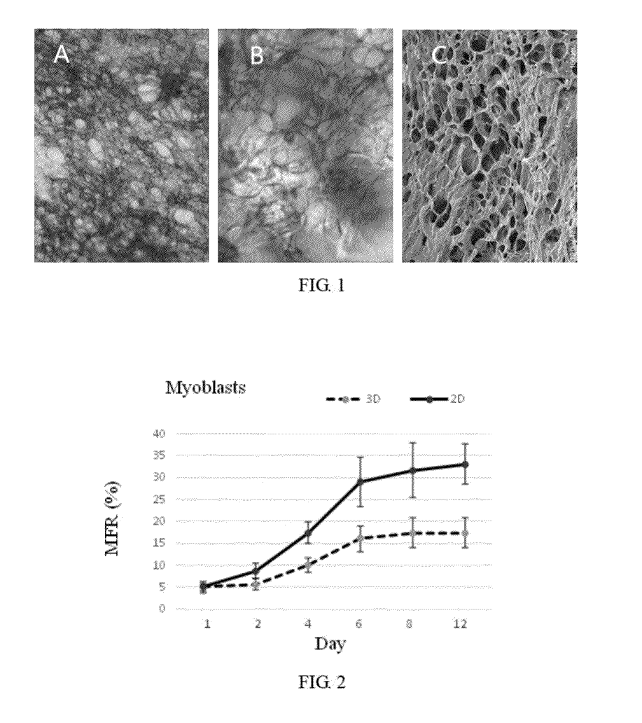

FIG. 1: freezing-dried 3D matrix scaffolds with different pore sizes (see the 400× optical microscope images (Olympus CX21, Japan) in FIGS. 1A-1B) and different conformations (see the SEM image (Philips XL20, Netherland) in FIG. 1C) were prepared from fibroin/chitosan solutions and crosslinking agents with different concentrations and ratios;

FIG. 2: MFRs of myoblasts in the 3D and 2D cultures both gradually increased with the time of cultivation and to 17% and 33%, respectively, on Day 12, wherein the myoblast MFR of the 3D culture showed a plateau from Day 6 to Day 12;

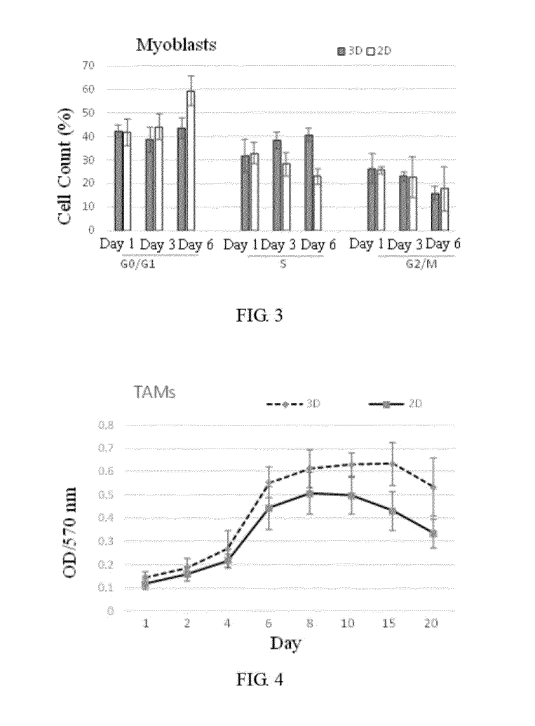

FIG. 3: percentages of the myoblasts in the synthesis phase (S-phase) in the 2D culture on Days 1, 3 and 6 were 33%, 28% and 23%, while those of the myoblasts in the 3D culture were 32%, 39% and 41%;

FIG. 4: OD values of TAMs in the 3D and 2D cultures both showed a significant rise from Day 4 of cultivation and reached 0.63 and 0.43, respectively, on Day 15, wherein the TAM OD value of the 3D culture showed a high plateau across Day 6 to Day 15;

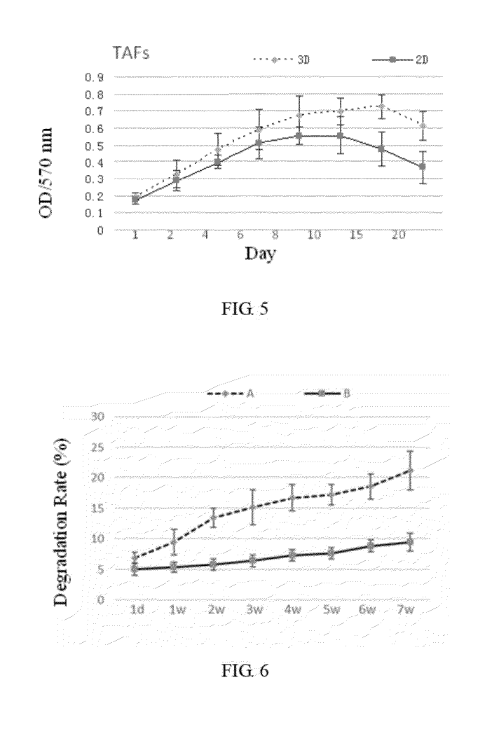

FIG. 5: OD values of TAFs in the 3D and 2D cultures both increased significantly from Day 2 of cultivation, and the TAF OD value of the 2D culture started to drop from the peak on Day 10, while this happened to that of the 3D culture on Day 15;

FIG. 6: the 3D scaffold made with the conventional fibroin solution significantly degraded by 21.2% after 7 weeks, while the degradation with time of that made with the fibroin powered prepared in accordance with the inventive method was mild and only 9.4% after 7 weeks; and

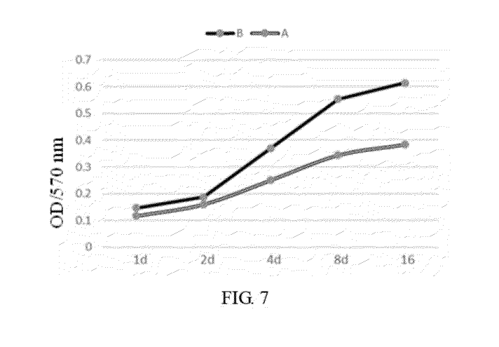

FIG. 7: as the conventional fibroin solution could not enable full performance of the intrinsic physicochemical properties of fibroin, the matrix scaffold made with this fibroin solution had a degradation rate increasing with its storage time, which deteriorated the cell proliferation ability of the scaffold, while the matrix scaffold made with the fibroin-like substance prepared according to the inventive method had a high stability and was thus advantageous for the adaptation and proliferation of the primary cells in the new environment, wherein with the use of the inventive matrix scaffold, the primary cells still maintained high proliferation ability even after two weeks, and wherein there were significant differences (P=0.031) between the two cases.

### DETAILED DESCRIPTION

This invention provides a 3D cell culture matrix scaffold resulting from a crosslinking reaction of a fibroin-like substance prepared in accordance with the invention, chitosan and crosslinking agents, wherein by means of changing concentrations and ratios of a fibroin-like substance/chitosan solution and the crosslinking agents, the 3D matrix scaffold can be made in different conformations and with different pore sizes which can be selected to be compatible with structural characteristics of in-vivo tissues where the cells to be cultured should grow. This 3D matrix provides a scaffold for seeded cells to adhere to and allows the cells to grow in a manner similar to as in the in-vivo microenvironment without suffering from the contact inhibition effect associated with the use of a 2D scaffold.

### EXAMPLE 1

### Matrix Scaffold for 3D Cell Cultivation and Method of its Preparation

A) Preparation of 1%-5% Fibroin Solution
1) Silk cocoons were cut into 1 cm² pieces which were then boiled in a 0.5% Na₂CO₃ solution for at least 1 hour while being completely immersed in the solution. This boiling process was repeated totally 2-3 times.
2) The resulting fibroin was rinsed first with natural water 2-3 times and then with deionized water twice. The rinsed fibroin was then dried.
3) The dried fibroin was added to a boiling 50% CaCl₂ solution (or 9 M LiBr solution), and the solution was then stirred until the fibroin was completely dissolved. Afterward, the whole was cooled to the room temperature and then filtered with a Buchner funnel.
4) The filtrate was collected and dialyzed in a dialysis bag against deionized water for 3-5 days, thereby producing a fibroin solution.
5) The fibroin solution was packed in a freezer bag and subjected therewith to freezing in a −20° C. refrigerator (12 hours) and then in a −80° C. refrigerator (6 hours) and to drying in a freezing dryer for at least 24 hours.
6) 10 g of the resulting fibroin powder was weighed and dissolved in a 9 M LiBr solution, wherein the dissolution is facilitated by a stirring action for 2 hours at the room temperature.
7) The resulting fibroin solution was then cooling to the room temperature and poured into a dialysis bag with a cutoff molecular weight of 3,500 Daltons. The solution in the bag was then dialyzed in a 4° C. refrigerator against deionized water for 3 days in order to remove low-molecular substances contained in the solution, wherein change of deionized water was conducted in every 3 hours.
8) The resulting fibroin solution was filled in another dialysis bag and concentrated in a polyethylene glycol 6000 powder. The concentrated solution was then collected and centrifuged at a speed of 3500 r/min for 15 minutes. The supernatant was then collected and stored in a 4° C. refrigerator selectively for a maximum period of one week.
9) Three labeled weighing bottles were successively cleaned, dried in a 60° C. constant temperature oven, sufficiently cooled and weighed. Their weights were denoted as M1. Afterward, each of the weighing bottles was filled with 10 ml of the fibroin solution, and their weights measured with the solution in were denoted as M2. The weighing bottles were then heated in the 60° C. constant temperature oven for 12 hours, and their weights after they are cooled were measured and denoted as M3. After that, a concentration of the fibroin solution was calculated according to the following equation: fibroin solution concentration (%)=(M3−M1)/(M2−M1)×100%.
10) In this example, the fibroin solution concentration was about 2%-3%.

B) Preparation of 1%-5% Chitosan Solution
1) A 1% glacial acetic acid solution was prepared by diluting 1 mL of glacial acetic acid to 100 ml. A pH of the glacial acetic acid solution was then adjusted to 4.6.
2) 3.1 g of chitosan (with a deacetylation of >90%) was weighed and dissolved in the glacial acetic acid solution.
3) In this example, a 1% chitosan solution was prepared.

C) Formation of Crosslinked Scaffold
1) The fibroin solution prepared in Section A) was mixed with the chitosan solution prepared in Section B).
2) The mixture was immersed in a 95% ethanol aqueous solution containing 50 mmol/l of EDC and 18 mmol/l of NHS, and the crosslinking reaction was maintained at 4° C. for 24 h.
3) A precursor fibroin/chitosan scaffold was obtained by subjecting a product of the reaction to pre-freezing in a −20° C. refrigerator for 24 h, then freezing in a −80° C. refrigerator for 24 h, and then freezing and drying in a freezing dryer for 48 h;
4) The precursor scaffold was immersed in anhydrous methanol mixed with a 10% NaOH solution (in a ratio by volume of 1:1) for 24 h, followed by rinsing thrice with deionized water, drying for 48 h in the freezing dryer and microscopic examination.
5) 3D matrix scaffolds prepared from fibroin/chitosan solutions and crosslinking agents with different concentrations and ratios were observed to have different pore sizes (see the optical microscope images (Olympus CX21, Japan) in FIGS. 1A and 1B) and different conformations (see the image in FIG. 1C, captured by a (Philips XL20, Netherland)).

### EXAMPLE 2

### Proliferation and Differentiation of Myoblasts in 3D Fibroin Culture Matrix

A) Isolation, Cultivation and Identification of Primary Myoblasts
1) A 15-week aborted embryo donated by a healthy woman without genetic history who terminated her pregnancy voluntarily was used.
2) Skeletal muscle tissue was harvested under sterile conditions, followed by removal of fasciae and blood vessels therefrom. Myoblasts were then isolated by repeated digestion with trypsin and collagenase and purified by differential adherence.
3) The purified myoblasts was cultured in a DMEM medium containing 10% fetal bovine serum (FBS) (as a growth medium (GM)) for 1 day and then in a DMEM containing 3% FBS (as a differentiation medium (DM)) for 6 days, followed by an observation with a phase-contrast inverted microscope for the formation of myotubes as well as immunohistochemical identification of myosin isoforms.

B) Differentiation of Myoblasts in 3D Fibroin Culture Matrix
1) 24-Well plates were respectively prepared for a test group and a control group, wherein the former was applied with the 3D fibroin matrix (referred to hereinafter as the “3D culture”) prepared in Example 1 and the latter was a common culture plate (referred to hereinafter as the “2D culture”).
2) The prepared myoblasts were inoculated in a dose of 5×10⁴/ml to each of the 24-well plates, followed by cultivation in the GM for one day and then in the DM for the rest days. Giemsa staining was performed on Days 1, 2, 4, 6 and 8 for observing fusion of myoblasts into myotubes.
3) MFR, a measure for differentiation of myoblasts, was defined as a ratio of the number of nuclei in myotubes to the number of all nuclei per unit area in the field-of-view. A higher MFR indicated more cells that had exited the cell cycle and were undergoing differentiation. The whole process was repeated three times, and the results were statistically analyzed.
4) As revealed in the results, MFRs of myoblasts in both the 3D and 2D cultures began to rise from Day 4. However, MFR of the 2D culture kept rising until Day 12, while that of the 3D culture showed a plateau from Day 6 to Day 12. MFRs of the 2D and 3D cultures on Day 12 were 17% and 33%, respectively (shown in FIG. 2).
5) This demonstrated that the 3D culture was capable of providing the myoblasts with a scaffold that closely resembled the in-vivo environment and was thus conducive to the proliferation of the myoblasts as well as the slow-down of their induced differentiation.

C) Cell Cycle of Myoblasts in 3D Fibroin Culture Matrix
1) With similarity to step 1) of Section B), a 24-well plate applied with the 3D fibroin matrix produced in Example 1 was prepared for a test group, and a common culture plate was prepared for a control group.
2) Similarly to step 2) of Section B), the prepared myoblasts were inoculated in a dose of 5×10⁴/ml to each of the 24-well plates, followed by cultivation in a DMEM medium containing 10% FBS for 1 day and then a DMEM containing 3% FBS.
3) 6 Days later, the myoblasts were collected, rinsed with phosphate-buffered saline (PBS) (5 min×2), and centrifuged at 1,000 rpm. Afterward, the supernatant was discarded, and the cells were resuspended in pre-heated 4° C. PBS, followed by slow addition of cold ethanol which resulted in a final concentration of 70%. The suspension was then maintained overnight.
4) The suspension was then centrifuged and mixed with the same volume of propidium iodide (PI) as a staining liquid at 4° C. for 30 minutes, followed by analysis using a flow cytometry (Coulter Elite, the United States). The whole process was repeated three times, and the results were statistically analyzed.
5) As revealed in the results, percentages of the myoblasts in the synthesis phase (S-phase) in the 2D culture on Days 1, 3 and 6 were 33%, 28% and 23%, while those of the myoblasts in the 3D culture were 32%, 39% and 41% (shown in FIG. 3).
6) This demonstrated that most of the myoblasts in the 2D culture had exited the cell cycle and were undergoing differentiation to their end cells, while a larger percentage of the myoblasts in the 3D culture was in their synthesis phase and most of them were undergoing proliferation, suggesting that the 3D culture matrix can be used in the cultivation of cells that were difficult to be cultured.

### EXAMPLE 3

### Cultivation and Proliferation of Tumor Stromal Cells Isolated from Tumor Tissue in 3D Fibroin Matrix

A) Isolation, Cultivation and Identification of TAMs
1) Fresh colon cancer tissue was cut into 2 mm pieces and then digested with PBS containing 0.3% collagenase at 37° C. into a cell suspension. The suspension was filtered with a 70 μm stainless steel mesh, followed by centrifugation and washing with PBS.
2) The washed cells were resuspended in a serum-free RPMI-1640 medium, and the whole was then positioned in a culture flask for 40 minutes, followed by identification of adherent TAMs by means of CD68 fluorescent staining

B) Isolation, Cultivation and Identification of TAFs
1) Fresh colon cancer tissue was cut into pieces and then seeded in a DMEM medium containing 15% FBS. Cells growing out from the tissue pieces 5 days later were isolated by digestion with trypsin and subcultured.
2) Purification was performed according to the expression and morphological characteristics of α-SMA, vimentin and desmin.

C) Proliferation of Stromal TAMs and TAFs in 3D Fibroin Culture Matrix
1) With similarity to step 1) of Section B) of Example 1, a 24-well plate applied with the 3D fibroin matrix produced in Example 1 was prepared for a test group, and a common culture plate was prepared for a control group.
2) The identified TAMs and TAFs isolated from the cancer tissue were inoculated in a dose of 5×10⁴/ml to each of the 24-well plates, followed by cultivation in a DMEM medium containing 10% FBS for 1 day and then a DMEM containing 5% FBS.
3) The culture medium was discarded on Days 1, 2, 4, 8 and 16, and each well was then added with 500 μl of 3-(4,5-dimethylthiazol-2-yl)-2,5-diphenyltetra-zolium bromide (MTT)-containing culture medium (100 μl of MTT plus 400 μl of culture medium), followed by shaking on a shaker for full dissolution of crystals.
4) Absorbance was measured in optical density (OD) at 570 nm with an enzyme-linked immunosorbent assay (ELISA) analyzer. Each measurement was repeated three times, and the measurement results were averaged.
5) As revealed in the results, TAMs in both of the 3D and 2D cultures showed a significant rise in OD value from Day 4 of cultivation. The TAM OD value rise of the 2D culture continued to Day 10, and became gradual decrease thereafter. The TAM OD value of the 3D culture showed a high plateau across Day 6 to Day 15. TAM OD values of the 3D and 2D cultures on Day 15 were 0.63 and 0.43, respectively (shown in FIG. 4). OD values of TAFs in the 3D and 2D cultures both increased significantly from Day 2 of cultivation. The TAF OD value of the 2D culture started to drop from the peak on Day 10, while this happened to that of the 3D culture on Day 15 (shown in FIG. 5).
6) This demonstrated that the inventive 3D matrix provided TAMs and TAFs with a scaffold that resembled the in-vivo environment, which was conducive to the proliferation and cultivation of the cells.

### EXAMPLE 4 (COMPARATIVE)

### Degradability Comparison of Matrix Scaffold Made with Fibroin-Like Substance Prepared by Inventive Method and that Made with Conventional Fibroin (CN103418029A)

A) A 20% fibroin solution was prepared in accordance with a method according to Embodiment 5 of the invention disclosed by CN103418029A.

B) A 3% fibroin solution was prepared in the manner described in Section A) of Example 1.

C) A 1%-5% chitosan solution was prepared in the manner described in Section B) of Example 1.

D) The two fibroin solutions were respectively used to prepare crosslinked scaffolds A and B, in the manner described in Section C) of Example 1. In other words, the scaffold A was a matrix scaffold made with the fibroin prepared according to the conventional method, while the scaffold B was a matrix scaffold made with a fibroin-like material prepared according to the inventive method.

E) Degradability of the scaffolds was measured in the fashion described below.
1) A simulated body fluid (SBF) served as an in-vitro environment for the degradation which is conducted at a constant temperature of 37° C.
2) 40 ml of sterile SBF was filled in a 50 ml plastic vial.
3) The scaffolds A and B were weighed (the weights were denoted as W₀) and placed in the SBF which was maintained at 37° C. in a constant humidity.
4) Weights of the scaffolds were measured on Days 1, 7, 14, 21, 28, 35, 42 and 49 and denoted as W₁ in order to calculate the degradation rates according to: Degradation Rate=(W₀−W₁)/W₀×100%. Each weight measurement was preceded by rinsing the scaffolds with deionized water and drying them in a 60° C. oven. Each of the scaffolds was measured with three samples, and the measurement results were obtained as mean values±standard deviations.

F) As revealed in the results shown in FIG. 6, the 3D scaffold made with the conventional fibroin solution significantly degraded by 21.2% after 7 weeks, while the degradation with time of that made with the fibroin powered prepared in accordance with the inventive method was mild and only 9.4% after 7 weeks, demonstrating an unsatisfactory stability of the former and a significant improvement in this regard in the latter.

### EXAMPLE 5 (COMPARATIVE)

### Cell Proliferation Ability Comparison of Matrix Scaffold Made with Fibroin-Like Substance Prepared by Inventive Method and that Made with Conventional Fibroin

A) A conventional fibroin solution was prepared in the same manner as described in Section A) of Example 4.

B) A 1%-5% fibroin solution was prepared in the same manner as described in Section A) of Example 1.

C) A 1%-5% chitosan solution was prepared in the manner described in Section B) of Example 1.

D) The two fibroin solutions were respectively used to prepare crosslinked scaffolds A and B two weeks after their preparation, in the same manner as described in Section C) of Example 1. In other words, the scaffold A was a matrix scaffold made with the fibroin prepared according to the conventional method, while the scaffold B was a matrix scaffold made with a fibroin-like material prepared according to the inventive method.

E) MTT assays were performed in the fashion described below to assess the proliferation of primary cells isolated from a colon cancer tissue in the 3D scaffolds A and B.
1) Necrotic and ulcerous lesions were removed from the tissue obtained from surgical or biopsy specimens. After immersion in PBS containing penicillin and streptomycin for 10-20 minutes, the tissue was placed in a serum-free preservation solution (DMEM/RPMI 1640 plus 10% penicillin/streptomycin) that was kept on ice.
2) The tissue was cut into pieces within PBS (containing 10% penicillin/streptomycin and 1% FBS) kept on ice.
3) Subsequently, the tissue, together with the PBS, was centrifuged at 1000 r/min for 5 minutes. The supernatant was removed, and the tissue was digested with collagenase and trypsin, following by incubation in a 37° C. incubator for about 1 hour which was shaken in every 10-15 minutes.
4) The whole was then centrifuged, and the supernatant was discarded. The resulting pellet was washed 2-3 times with PBS by low-speed centrifugation, and the supernatant was again discarded. At last, the resulting pellet was washed once by centrifugation with culture medium, suspended in complete medium and dispersed by pipetting, thereby resulting in a cell suspension which was subsequently inoculated to DMED (or RPMI 1640) medium containing 10% FBS and cultured at 37° C. in a 5% CO₂ atmosphere in flasks (or dishes).
5) The culture medium was discarded on Days 1, 2, 4, 8 and 16, and each well was then added with 500 μl of MTT-containing culture medium (100 μl of MTT plus 400 μl of culture medium), followed by shaking on a shaker for full dissolution of crystals.
6) Absorbance was measured in OD at 570 nm with an ELISA analyzer. Each measurement was repeated three times, and the measurement results were averaged

F) As revealed in the results shown in FIG. 7, as the conventional fibroin solution could not enable full performance of the intrinsic physicochemical properties of fibroin, the matrix scaffold made with this fibroin solution had a degradation rate increasing with its storage time, which deteriorated the cell proliferation ability of the scaffold. In contrast, the matrix scaffold made with the fibroin-like substance prepared according to the inventive method had a high stability and was thus advantageous for the adaptation and proliferation of the primary cells in the new environment. With the use of the inventive matrix scaffold, the primary cells still maintained high proliferation ability even after two weeks. There were significant differences (P=0.031) between the two cases.

The foregoing examples have been presented only for explaining the principles of the present invention, thereby enabling those skilled in the art to understand and carry out the invention. They are not intended to limit the scope of the invention in any way. All equivalent variations made in light of the above teachings as well as the use thereof in different applications are within the scope of the invention.

1. A matrix scaffold for three-dimensional (3D) cell cultivation, resulting from a crosslinking reaction of a fibroin-like substance, chitosan and crosslinking agents, wherein the fibroin-like substance is prepared by obtaining a fibroin powder through subjecting silk cocoons or filaments to degumming, dissolution, dialysis and drying processes and performing the following steps on the fibroin powder:
1) dissolution of the fibroin powder in a lithium bromide solution;
2) dialysis of the fibroin solution resulting from step 1) in a dialysis bag with a cutoff molecular weight of 3,500 Daltons; and
3) concentration of the fibroin solution that has been dialyzed in step 2) by positioning the dialysis bag containing the fibroin solution in a polyethylene glycol 6000 powder, centrifugation of the concentrated fibroin solution, and obtainment of the supernatant as the fibroin-like substance.

2. The matrix scaffold for 3D cell cultivation of claim 1, wherein the crosslinking agents are 1-ethyl-3-(3-dimethylaminopropyl)carbodiimide (EDC) and N-hydroxysuccinimide (NHS).

3. The matrix scaffold for 3D cell cultivation of claim 1, wherein the matrix scaffold is obtained through gradient freezing of a product of the crosslinking reaction of the fibroin-like substance, chitosan and crosslinking agents, wherein the gradient freezing comprises:
1) obtaining a precursor 3D scaffold by subjecting the product to pre-freezing in a −20° C. refrigerator for 12-48 h, then freezing in a −80° C. refrigerator for 12-48 h, and then freezing and drying in a freezing dryer for 24-72 h; and
2) immersing the precursor 3D scaffold obtained in step 1) in anhydrous methanol mixed with a 10% sodium hydroxide solution (in a ratio by volume of 1:1) for 12-48 h, followed by rinsing with deionized water and drying for 24-72 h in the freezing dryer.

4. The matrix scaffold for 3D cell cultivation of claim 1, wherein various conformations and pore sizes of the matrix scaffold can be achieved by adjusting amount(s) of the fibroin-like substance, chitosan and/or crosslinking agents.

5. The matrix scaffold for 3D cell cultivation of claim 1, wherein the matrix scaffold is prepared by a method comprising the steps of:
A) preparation of a solution of a fibroin-like substance, including:
1) cutting silk cocoons into small pieces and boiling the pieces in a 0.5% sodium carbonate solution 2-3 times, following by rinsing with deionized water and drying;
2) dissolving, with stirring, the dried fibroin resulting from step 1) in a boiling 50% calcium chloride solution and filtering the whole after being cooled;
3) obtaining a fibroin solution by dialyzing the filtrate contained in a dialysis bag against deionized water and subsequently a fibroin powder by subjecting the fibroin solution packaged in a freezer bag successively to freezing in a −20° C. refrigerator and then in a −80° C. refrigerator and drying in a freezing dryer;
4) weighing 10 g of the fibroin powder obtained in step 3) and dissolving the fibroin powder in a 9 M lithium bromide solution, wherein the dissolution is facilitated by a stirring action at room temperature;
5) cooling the fibroin solution resulting from step 4) to the room temperature and then dialyzing the fibroin solution in a dialysis bag with a cutoff molecular weight of 3,500 Daltons for 2-4 days in order to remove low-molecular substances contained in the fibroin solution; and
6) concentrating the fibroin solution resulting from step 5) by positioning the dialysis bag containing the fibroin solution in a polyethylene glycol 6000 powder, centrifuging the concentrated fibroin solution and collecting the supernatant as the solution of the fibroin-like substance,
B) preparation of a chitosan solution, including:
1) preparing a 1% glacial acetic acid solution by diluting 1 mL of glacial acetic acid to 100 ml and adjusting a pH of the glacial acetic acid solution to 4.6; and
2) dissolving an amount of chitosan (with a deacetylation of >90%) in the glacial acetic acid solution, thereby forming the chitosan solution, and
C) formation of a crosslinked scaffold, including
1) mixing the solution of the fibroin-like substance prepared in step A) with the chitosan solution prepared in step B);
2) immersing the mixture in a 95% ethanol aqueous solution containing 50 mmol/l of EDC and 18 mmol/l of NHS and maintaining a crosslinking reaction thereof at 4° C. for 24 h;
3) obtaining a precursor 3D scaffold by subjecting a product of the crosslinking reaction in step 2) to pre-freezing in a −20° C. refrigerator for 24 h, then freezing in a −80° C. refrigerator for 24 h, and then freezing and drying in a freezing dryer for 48 h; and
4) obtaining the matrix scaffold for 3D cell cultivation by immersing the scaffold obtained in step 3) in anhydrous methanol mixed with a 10% sodium hydroxide solution (in a ratio by volume of 1:1) for 24 h, followed by rinsing thrice with deionized water and drying for 48 h in the freezing dryer.

6. The matrix scaffold for 3D cell cultivation of claim 5, wherein the solution of the fibroin-like substance has a concentration of 1%-5%, and the chitosan solution has a concentration of 1%-5%.

7. Use of a matrix scaffold for 3D cell cultivation for the in-vitro differentiation and proliferation of cells, reconstruction of tissues and organs, or screening of anticancer drugs, wherein the matrix scaffold for 3D cell cultivation is resulted from a crosslinking reaction of a fibroin-like substance, chitosan and crosslinking agents, wherein the fibroin-like substance is prepared by obtaining a fibroin powder through subjecting silk cocoons or filaments to degumming, dissolution, dialysis and drying processes and performing the following steps on the fibroin powder:
1) dissolution of the fibroin powder in a lithium bromide solution;
2) dialysis of the fibroin solution resulting from step 1) in a dialysis bag with a cutoff molecular weight of 3,500 Daltons; and
3) concentration of the fibroin solution that has been dialyzed in step 2) by positioning the dialysis bag containing the fibroin solution in a polyethylene glycol 6000 powder, centrifugation of the concentrated fibroin solution, and obtainment of the supernatant as the fibroin-like substance.

8. The use of claim 7, for the cultivation of stem cells, engineering of tumor microenvironments, screening of antitumor drugs, or engineering of tissues and organs.

9. The use of claim 7, for the in-vitro differentiation of myoblasts isolated from embryonic tissue or tumor associated macrophages (TAMs) or tumor-associated fibroblasts (TAFs) isolated from tumor tissues.

10. A method of in-vitro cell proliferation, comprising using the matrix scaffold as defined in claim 1 as a scaffold for 3D cell cultivation.

11. The use of claim 7, wherein the crosslinking agents are 1-ethyl-3-(3-dimethylaminopropyl)carbodiimide (EDC) and N-hydroxysuccinimide (NHS).

12. The use of claim 7, wherein the matrix scaffold is obtained through gradient freezing of a product of the crosslinking reaction of the fibroin-like substance, chitosan and crosslinking agents, wherein the gradient freezing comprises:
1) obtaining a precursor 3D scaffold by subjecting the product to pre-freezing in a −20° C. refrigerator for 12-48 h, then freezing in a −80° C. refrigerator for 12-48 h, and then freezing and drying in a freezing dryer for 24-72 h; and
2) immersing the precursor 3D scaffold obtained in step 1) in anhydrous methanol mixed with a 10% sodium hydroxide solution (in a ratio by volume of 1:1) for 12-48 h, followed by rinsing with deionized water and drying for 24-72 h in the freezing dryer.

13. The use of claim 7, wherein various conformations and pore sizes of the matrix scaffold can be achieved by adjusting amount(s) of the fibroin-like substance, chitosan and/or crosslinking agents.

14. The use of claim 7, wherein the matrix scaffold is prepared by a method comprising the steps of:
A) preparation of a solution of a fibroin-like substance, including:
1) cutting silk cocoons into small pieces and boiling the pieces in a 0.5% sodium carbonate solution 2-3 times, following by rinsing with deionized water and drying;
2) dissolving, with stirring, the dried fibroin resulting from step 1) in a boiling 50% calcium chloride solution and filtering the whole after being cooled;
3) obtaining a fibroin solution by dialyzing the filtrate contained in a dialysis bag against deionized water and subsequently a fibroin powder by subjecting the fibroin solution packaged in a freezer bag successively to freezing in a −20° C. refrigerator and then in a −80° C. refrigerator and drying in a freezing dryer;
4) weighing 10 g of the fibroin powder obtained in step 3) and dissolving the fibroin powder in a 9 M lithium bromide solution, wherein the dissolution is facilitated by a stirring action at room temperature;
5) cooling the fibroin solution resulting from step 4) to the room temperature and then dialyzing the fibroin solution in a dialysis bag with a cutoff molecular weight of 3,500 Daltons for 2-4 days in order to remove low-molecular substances contained in the fibroin solution; and
6) concentrating the fibroin solution resulting from step 5) by positioning the dialysis bag containing the fibroin solution in a polyethylene glycol 6000 powder, centrifuging the concentrated fibroin solution and collecting the supernatant as the solution of the fibroin-like substance,
B) preparation of a chitosan solution, including:
1) preparing a 1% glacial acetic acid solution by diluting 1 mL of glacial acetic acid to 100 ml and adjusting a pH of the glacial acetic acid solution to 4.6; and
2) dissolving an amount of chitosan (with a deacetylation of >90%) in the glacial acetic acid solution, thereby forming the chitosan solution, and
C) formation of a crosslinked scaffold, including
1) mixing the solution of the fibroin-like substance prepared in step A) with the chitosan solution prepared in step B);
2) immersing the mixture in a 95% ethanol aqueous solution containing 50 mmol/l of EDC and 18 mmol/l of NHS and maintaining a crosslinking reaction thereof at 4° C. for 24 h;
3) obtaining a precursor 3D scaffold by subjecting a product of the crosslinking reaction in step 2) to pre-freezing in a −20° C. refrigerator for 24 h, then freezing in a −80° C. refrigerator for 24 h, and then freezing and drying in a freezing dryer for 48 h; and
4) obtaining the matrix scaffold for 3D cell cultivation by immersing the scaffold obtained in step 3) in anhydrous methanol mixed with a 10% sodium hydroxide solution (in a ratio by volume of 1:1) for 24 h, followed by rinsing thrice with deionized water and drying for 48 h in the freezing dryer.

15. The use of claim 14, wherein the solution of the fibroin-like substance has a concentration of 1%-5%, and the chitosan solution has a concentration of 1%-5%.

16. The method of claim 10, wherein the crosslinking agents are 1-ethyl-3-(3-dimethylaminopropyl)carbodiimide (EDC) and N-hydroxysuccinimide (NHS).

17. The method of claim 10, wherein the matrix scaffold is obtained through gradient freezing of a product of the crosslinking reaction of the fibroin-like substance, chitosan and crosslinking agents, wherein the gradient freezing comprises:
1) obtaining a precursor 3D scaffold by subjecting the product to pre-freezing in a −20° C. refrigerator for 12-48 h, then freezing in a −80° C. refrigerator for 12-48 h, and then freezing and drying in a freezing dryer for 24-72 h; and
2) immersing the precursor 3D scaffold obtained in step 1) in anhydrous methanol mixed with a 10% sodium hydroxide solution (in a ratio by volume of 1:1) for 12-48 h, followed by rinsing with deionized water and drying for 24-72 h in the freezing dryer.

18. The method of claim 10, wherein various conformations and pore sizes of the matrix scaffold can be achieved by adjusting amount(s) of the fibroin-like substance, chitosan and/or crosslinking agents.

19. The method of claim 10, wherein the matrix scaffold is prepared by a method comprising the steps of:
A) preparation of a solution of a fibroin-like substance, including:
1) cutting silk cocoons into small pieces and boiling the pieces in a 0.5% sodium carbonate solution 2-3 times, following by rinsing with deionized water and drying;
2) dissolving, with stirring, the dried fibroin resulting from step 1) in a boiling 50% calcium chloride solution and filtering the whole after being cooled;
3) obtaining a fibroin solution by dialyzing the filtrate contained in a dialysis bag against deionized water and subsequently a fibroin powder by subjecting the fibroin solution packaged in a freezer bag successively to freezing in a −20° C. refrigerator and then in a −80° C. refrigerator and drying in a freezing dryer;
4) weighing 10 g of the fibroin powder obtained in step 3) and dissolving the fibroin powder in a 9 M lithium bromide solution, wherein the dissolution is facilitated by a stirring action at room temperature;
5) cooling the fibroin solution resulting from step 4) to the room temperature and then dialyzing the fibroin solution in a dialysis bag with a cutoff molecular weight of 3,500 Daltons for 2-4 days in order to remove low-molecular substances contained in the fibroin solution; and
6) concentrating the fibroin solution resulting from step 5) by positioning the dialysis bag containing the fibroin solution in a polyethylene glycol 6000 powder, centrifuging the concentrated fibroin solution and collecting the supernatant as the solution of the fibroin-like substance,
B) preparation of a chitosan solution, including:
1) preparing a 1% glacial acetic acid solution by diluting 1 mL of glacial acetic acid to 100 ml and adjusting a pH of the glacial acetic acid solution to 4.6; and
2) dissolving an amount of chitosan (with a deacetylation of >90%) in the glacial acetic acid solution, thereby forming the chitosan solution, and
C) formation of a crosslinked scaffold, including
1) mixing the solution of the fibroin-like substance prepared in step A) with the chitosan solution prepared in step B);
2) immersing the mixture in a 95% ethanol aqueous solution containing 50 mmol/l of EDC and 18 mmol/l of NHS and maintaining a crosslinking reaction thereof at 4° C. for 24 h;
3) obtaining a precursor 3D scaffold by subjecting a product of the crosslinking reaction in step 2) to pre-freezing in a −20° C. refrigerator for 24 h, then freezing in a −80° C. refrigerator for 24 h, and then freezing and drying in a freezing dryer for 48 h; and
4) obtaining the matrix scaffold for 3D cell cultivation by immersing the scaffold obtained in step 3) in anhydrous methanol mixed with a 10% sodium hydroxide solution (in a ratio by volume of 1:1) for 24 h, followed by rinsing thrice with deionized water and drying for 48 h in the freezing dryer.

20. The method of claim 19, wherein the solution of the fibroin-like substance has a concentration of 1%-5%, and the chitosan solution has a concentration of 1%-5%.
